(19) United States
(12) Patent Application Publication
(10) Pub. No.: US 2025/0065146 A1
(43) Pub. Date: Feb. 27, 2025

(54) APPARATUS AND METHODS FOR CONTROLLING AXIAL GROWTH WITH APPLICATION OF WHITE LIGHT

(71) Applicant: MENICON CO., LTD., Nagoya (JP)

(72) Inventor: Stephen D. Newman, Singapore (SG)

(21) Appl. No.: 18/815,763

(22) Filed: Aug. 26, 2024

Related U.S. Application Data
(60) Provisional application No. 63/578,934, filed on Aug. 25, 2023.

(51) Int. Cl. A61N 5/06

(52) U.S. Cl. CPC A61N 5/0613; A61N 2005/0626; A61N 2005/0648; A61N 2005/0663; A61N 2005/0664

(57) ABSTRACT

Systems, apparatuses, and methods for applying white light to a user's eyelids to control myopia are disclosed. In an example, a method for controlling axial length of a user's eye includes applying a white light to the user's eyelid; detecting an effect of the applied white light on the axial length of the user's eye; and adjusting the applied white light based on the detected effect.

### CROSS-REFERENCE TO RELATED APPLICATION

This application claims the benefit of priority to U.S. Provisional Application No. 63/578,934 entitled “Apparatus and Methods for Controlling Axial Growth with Application of White Light,” filed 25 Aug. 2023, the disclosure of which is hereby incorporated by reference in its entirety.

### BACKGROUND

Emmetropia is a state of vision where a viewer sees objects clearly at both near and far distances. The cornea and crystalline lens collectively focus the light entering the eye to the central regions of the retina. Emmetropia is achieved when the collective refractive powers of the cornea and crystalline lens focus light exactly onto the central portion of the retina.

Myopia is a vision condition where objects near a viewer appear clear, but objects that are spaced further from the viewer are progressively blurred. Myopia is sometimes referred to as being nearsighted. Myopia can be caused by any number of conditions and reasons. A significant factor for many cases of myopia includes an elongated axial length of the eye. Myopia occurs when the focal point of the focused light entering the eye is formed in front of the retina. In other words, the focus of the light rays entering the eye converges short of the retina.

Another condition that is affected by the eye's axial length is hyperopia. This condition causes the viewer to see objects at a distance clearly, while the objects close to the viewer are progressively burred. While this condition can occur for multiple reasons as well, a person typically has hyperopia if the focal point of the focused light entering the eye is formed behind the retina.

The axial length of the eye grows as children age. As young people begin their young adulthood years, the eye generally stops growing and the axial length of the eye becomes more permanent. Thus, if the growth of the eye's axial length can be controlled during a child's youth, myopia or hyperopia can be reduced or even eliminated in the child's adulthood years. What is needed is an apparatus, system, and method for controlling the growth of the eye's axial length during any stage of life where the axial length of the eye is capable of growing.

Some apparatuses, systems, and methods for controlling the growth of an eye's axial length have been proposed. For example, systems and apparatuses that irradiate a subject's eye with red or near-infrared light, either directly or through the subject's eyelids, have been proposed. However, these methods for controlling the growth of the eye's axial length can cause retinal burn, overexposure, light exposure shock, require user compliance throughout the day (e.g., multiple times per day), and the like. What is further needed is an apparatus, system, and method for controlling the growth of the eye's axial length that is safe and convenient to use, providing improved outcomes and subject compliance.

### SUMMARY

A number of representative embodiments are provided to illustrate the various features, characteristics, and advantages of the disclosed subject matter. It should be understood that the features, characteristics, advantages, and the like described in connection with one embodiment can be used separately or in various combinations and sub-combinations with other features described in connection with other embodiments.

According to one exemplary embodiment, a method for controlling axial length of a user's eye can include applying a dose of white light to the user's eyelid, detecting an effect of the applied white light on the user's eye, and adjusting the applied white light based on the detected effect.

In some examples, applying the dose of white light to the user's eyelid applies a dose red light to the user's eye having a wavelength in a range from 600 nm to 650 nm. In some examples the detected effect of the applied white light is the axial length of the user's eye. The method can include applying a white light with a ramped-up intensity prior to applying the dose of white light. The method can also include applying a white light with a ramped-down intensity subsequent to applying the dose of white light. In some examples, the method can further include detecting a diameter of a pupil of the user's other eye as the white light is applied to the user's eye and can also include adjusting the applied white light based on the detected pupil diameter of the user's eye.

In some examples, the method can further include detecting pressure applied to the user's eye from a white light source during the application of the white light and can include adjusting a tightness of a strap configured to retain the white light source on the user's head in response to the detected pressure. In some examples the white light is applied to the user while the user is in a sleeping state. Additionally, the applied dose of white light can have an illuminance in a range from 30,000 lux to 35,000 lux. In some examples the applied dose of white light is applied for a duration in a range from 2 minutes to 3 minutes.

In another embodiment a system configured to apply red light to a user's eye through the user's eyelid includes a white light source, a sensor configured to detect an axial length of a user's eye, and a controller configured to vary the white light source based on the detected axial length of the user's eye. In some examples the sensor uses optical low coherence reflectometry to detect the axial length of the user's eye. In some examples the sensor uses optical coherence tomography (OCT) to detect the axial length of the user's eye. In some examples the sensor is configured to direct infrared light towards the user's eye.

In some examples, the system further includes a device housing that houses the white light source, the sensor, and the controller, and a strap configured to retain the system on the user's head. The system can further include a pressure sensor configured to detect pressure applied by the device housing to the user's eyes, wherein the strap is adjustable based on the pressure detected by the pressure sensor.

In other embodiments, an apparatus includes a device housing having a white light source configured to apply a white light to a user's eyelid, and a sensor configured to detect a characteristic of an eye. The apparatus can further include a strap coupled to the device housing and configured to retain the device housing on a user's head.

In some examples of the apparatus, the sensor can be a pressure sensor, the sensor can be configured to detect pressure applied to an eye by the apparatus, and the strap can be configured to be adjusted based on the detected pressure.

The Summary is provided to introduce a selection of concepts in a simplified form that are further described below in the Detailed Description. The Summary and the Background are not intended to identify key concepts or essential aspects of the disclosed subject matter, nor should they be used to constrict or limit the scope of the claims. For example, the scope of the claims should not be limited based on whether the recited subject matter includes any or all aspects noted in the Summary and/or addresses any of the issues noted in the Background.

### BRIEF DESCRIPTION OF THE DRAWINGS

The accompanying drawings illustrate various embodiments of the principles described herein and are part of the specification. The illustrated embodiments are merely examples and do not limit the scope of the claims.

FIG. 1 is a side schematic view of a system for directing white light through an eyelid into an eye.

FIG. 2 is a front view of an apparatus for directing white light through a user's eyelid into the user's eye mounted on the user's head.

FIG. 3 is a flow diagram of a method for directing white light through a user's eyelid into the user's eye.

FIG. 4 is a graph showing light transmittance through a user's eyelid into the user's eye.

FIG. 5 is a graph showing light transmittance through a 781 Terry Red filter.

FIG. 6A is a graph showing white light transmittance through a user's eyelid by intensity interaction on pupil size change.

FIG. 6B is a graph showing red light transmittance through a user's open eye by intensity interaction on pupil size change.

FIG. 7 is a graph showing axial length changes averaged across three levels of light intensity and across 0-minute and 10-minutes post exposure times.

FIG. 8 is a graph showing axial length changes for white light (through the eyelid) and red light (broadband) open eye at low, mid and high intensities averaged for 0 minute and 10 minutes post-exposure.

FIG. 9A is a graph showing axial length changes for white light (through the eyelid) at low, mid, and high intensities at 0 minute and 10 minutes post-exposure.

FIG. 9B is a graph showing axial length changes for red light (broadband) on an open eye at low, mid, and high intensities at 0 minute and 10 minutes post-exposure.

FIG. 10 is a graph showing changes in subfoveal choroidal thickness (SFCT) for white light (through the eyelid) versus red light (broadband) open eye at three intensities and averaged across 0-minute and 10-minutes post exposure times.

FIG. 11A is a graph showing changes in subfoveal choroidal thickness (SFCT) for white light (through the eyelid) at three intensities at 0-minute and 10-minutes post exposure times.

FIG. 11B is a graph showing changes in subfoveal choroidal thickness (SFCT) for red light (broadband) at three intensities at 0-minute and 10-minutes post exposure times.

FIG. 12 is a graph showing changes in foveal luminal thickness (FLT) for white light (through the eyelid) versus red light (broadband) open eye at three intensities and averaged across 0-minute and 10-minutes post exposure times.

FIG. 13 is a graph showing changes in foveal stromal thickness (FST) for white light (through the eyelid) versus red light (broadband) open eye at three intensities and averaged across 0-minute and 10-minutes post exposure times.

Throughout the drawings, identical reference numbers designate similar, but not necessarily identical, elements.

### DETAILED DESCRIPTION

The growth of an eye's axial length can be affected by light received in the retina. Specific wavelengths of light can be used to balance the axial length of the eye with the collective focusing ability of the cornea and crystalline lens. The eye uses the focal point of incident light focused on the retina to determine when the eye's axial length is balanced. There is a 1.7-2 D difference in focus across the visible spectrum, resulting in short wavelengths focusing in front of the retina and long wavelengths focusing behind the retina. This difference in focus can be used to apply light of specific wavelengths to the eye in order to provide directional cues to the eye to increase the eye's axial length, or to stop increasing the eye's axial length or even decrease the eye's axial length. These changes in axial length can be accompanied by changes in the choroid thickness. For example, light received in the retina that causes the eye to stop axial growth or decrease axial growth can cause the choroid to thicken, while light received in the retina that causes the eye to continue axial growth can cause the choroid to thin.

Exposure of the eye to red light or near infrared light (e.g., light having wavelengths in a range of about 600 nm to about 680 nm) has been shown to reduce axial eye growth in children in a number of clinical trials. The mechanism of action of red-light therapy could relate to the longitudinal chromatic aberration of the eye, with red light in focus on the retina providing a signal that the eye is already too long and therefore providing a “stop” signal. Alternately or additionally, red light therapy may stimulate increased blood flow in the choroid, resulting in a thicker choroid, which may produce a “stop” signal that reduces scleral hypoxia. Both of these mechanisms can be used to reduce myopia in subjects.

Red light can be applied to a user's eyes by applying white light to the user's eyelids. When white light is applied to a user's eyelids, transmittance through the user's eyelids is highest for light wavelengths at the red end of the visible spectrum. Light transmittance through the eyelids is relatively consistent between wavelengths of 600 nm and 650 nm and the transmittance in this red wavelength range is in a range from about 5% to about 10% of incident light. Thus, red light can be applied to a subject's eyes by irradiating the user's eyelids with white light.

Applying white light to a user's eyelids, rather than applying red light directly to a user's eyes has several benefits. White light is cooler than red light, which reduces the likelihood of retinal burn. Moreover, the user's eyelids act to diffuse light incident on the user's eyes, which also reduces adverse effects. The present disclosure provides systems, apparatuses, and methods that can be used to apply white light to a user's eyelids in order to control axial length of the user's eyes.

In addition to benefits resulting from using white light applied to a user's eyelids rather than applying red light directly to the user's eyes, the present disclosure provides a ramp-up period prior to administering a full dose of white light to a user. The ramp-up period increases the intensity of white light applied to the user's eyes, which prevents the user from experiencing light shock. The apparatus and system can include a device housing and a strap that are secured to a user's head and can apply doses of white light to a user while the user is sleeping. This allows for the ramp-up period to be provided without the user using additional time during the day, allows for multiple doses to be administered through the night as the user is sleeping, and allows for measurements of the user's eye to be taken prior to and after administering a dose of white light. The housing can include sensors that measure the user's eye prior to, during, and subsequent to administering the dose of white light, and the measurements can be used to alter current and subsequent doses of white light applied to the user. The strap and/or housing can include pressure sensors, which can be used to adjust tightness of the strap in order to securely and comfortably retain the apparatus on the user's head. Users only have to don the apparatus once daily when they go to sleep, less than requirements for previous devices, benefitting user compliance.

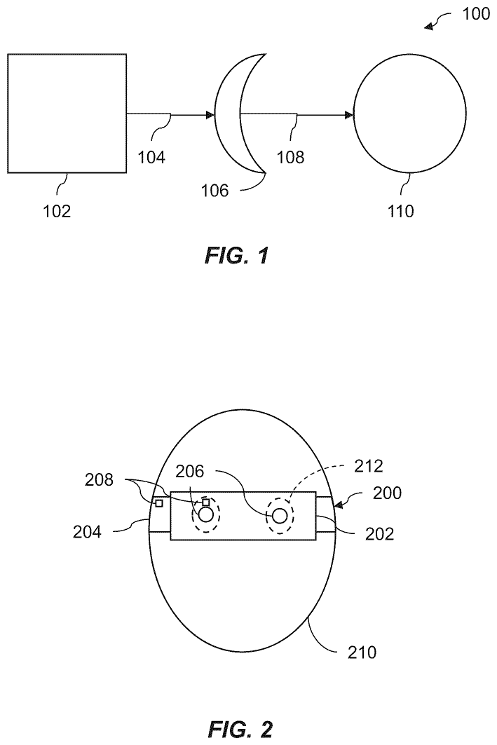

FIG. 1 illustrates a system 100 for administering a dose of white light to a user. The system 100 includes a white light source 102, an eyelid 106, and an eye 110. As illustrated in FIG. 1, white light 104 is generated by the white light source 102. As a dose of the white light 104 is applied to the eyelid 106, some of the white light 104 is absorbed by the eyelid 106. Light having red wavelengths (e.g., light with wavelengths in a range from about 600 nm to about 650 nm or in a range from about 600 nm to about 680 nm) has the highest transmittance through the eyelid 106, with a transmittance of about 5%. Thus, red light 108 passes through the eyelid 106 to the eye 110.

The luminance of the red light 108 that passes through the eyelid 106 can be about 5% of the luminance of the white light 104 applied to the eyelid. In some examples, the white light 104 can be applied to the eyelid 106 at a luminance of about 1,000 lux, about 10,000 lux, about 32,000 lux, in a range from about 1,000 lux to about 50,000 lux, in a range from about 1,000 lux to about 5,000 lux, in a range from about 5,000 lux to about 15,000 lux, in a range from about 25,000 lux to about 35,000 lux, or the like. The red light 108 applied to the eye 110 can be applied at a luminance of about 50 lux, about 500 lux, about 1,600 lux, in a range from about 50 lux to about 2,500 lux, in a range from about 50 lux to about 250 lux, in a range from about 250 lux to about 750 lux, in a range from about 1,250 lux to about 1,750 lux, or the like. The white light 104 can be applied to the eyelid 106 for a period of about 3 minutes, a period in a range from about 2 minutes to about 3 minutes, or the like.

FIG. 2 illustrates an apparatus 200 for applying a dose of white light to a user's eyelids 212. As illustrated in FIG. 2, the apparatus 200 includes a housing 202 and a strap 204. The housing 202 can house various electronics of the apparatus 200, including white light sources 206, a sensor 208, a battery, control buttons, a controller, and the like.

In the example of FIG. 2, a white light source 206 is provided adjacent to each of the user's eyelids 212. However, multiple white light sources 206 can be provided adjacent to each of the user's eyelids 212, or a single white light source 206 can be provided adjacent to a single user's eyelid 212, depending on the user's requirements. The white light sources 206 can include laser diodes (LDs), light-emitting diodes (LEDs), bulbs, or the like. A controller can be coupled to the white light sources 206 to alter the intensity, wavelength, bandwidth, application time, and the like of the white light sources 206. The controller can also control ramp-up, wind-down, and white light exposure periods of the white light sources 206.

A sensor 208 can be provided adjacent to each of the user's eyelids 212, or adjacent to one of the user's eyelids 212. The sensor 208 can be used to measure various features of the user's eyes, such as axial length, pupil diameter, choroid thickness, and the like. The sensor can use optical low coherence reflectometry, optical coherence tomography (OCT), or the like to detect features of the user's eyes. The controller can adjust the white light sources 206 based on measurements detected by the sensor 208, thereby customizing the dose of white light applied to the user's eyelids 212 based on the effect of the white light on the user's eyes.

The strap 204 is configured to retain the housing 202 on a user's head 210 such that the white light sources 206 are retained in desired positions adjacent to the user's eyelids 212. The strap 204 can include a sensor 208, such as a pressure sensor, and can adjust tension applied to the user's head and eyes based on the measurement of the pressure sensor. The sensor 208 included in the device housing can also be a pressure sensor, and tension can be adjusted in the strap 204 based on pressure applied to the user's eyes. The strap 204 can be adjusted to apply sufficient pressure to retain the apparatus 200 on the user's head 210, even as the user moves throughout sleep, while still remaining comfortable.

In some examples, the apparatus 200 of FIG. 2 may be a handheld apparatus, a desk-mounted apparatus, or the like, and the strap 204 can be omitted. In some examples, the apparatus 200 may be used while a user is awake. In some examples, the apparatus 200 may be a portable apparatus that can be set on a desktop or the like. The user can align their eyes with the white light sources 206, and white light can be applied to the user's eyelids 212 as the user is awake.

In some examples, a white light source 206 can be provided adjacent to each of a user's eyes, or a white light source 206 can be provided adjacent to one of the user's eyes, and a sensor 208 can be provided adjacent to the other user's eye. One or more sensors 208 and/or white light sources 206 can be provided adjacent to each of the user's eyes.

In some examples, the sensors 208 can be used to detect a pupil size of a user's open eye. For example, white light can be applied to one of the user's closed eyelids 212 through a white light source 206 adjacent to the user's closed eyelid 212, and a sensor 208 adjacent to the user's other open eye can detect a pupil size of the user's open eye. The pupils of a user's eyes both contract or dilate simultaneously such that measuring one eye's pupil size is a close estimation for the other's eye's pupil size.

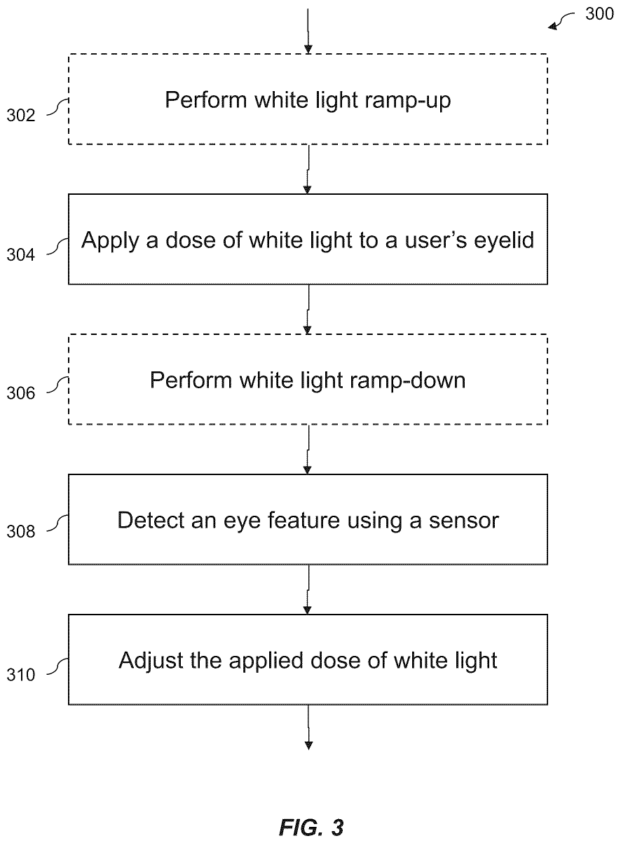

FIG. 3 illustrates a method 300 for applying white light to a user's eyelids. The method 300 can be used to control axial length of the user's eyes. In some examples, the method 300 can be used to apply red light indirectly to the user's eyes by applying white light directly to the user's eyelids, in a system that is the same as or similar to the system 100 discussed above with respect to FIG. 1. The various steps of the method 300 can be performed by an apparatus the same as or similar to the apparatus 200, discussed above with respect to FIG. 2.

The method 300 includes performing a white light ramp-up process at step 302. The intensity of white light applied to the user's eyelids during the white light ramp-up process can be varied (e.g., increased) continuously, or in a stepped manner. During the white light ramp-up process, the intensity of white light applied to a user's eyelids is increased over a period of time. The white light ramp-up process can have a duration in a range from about 1 minute to about 10 minutes, in a range from about 2 minutes to about 5 minutes, about 2 minutes, about 3 minutes, or about 4 minutes, or the like. In some examples, wavelengths of the white light applied to the user's eyelids can be varied during the white light ramp-up process of step 302. For example, shorter wavelengths of light can be applied at the beginning of the white light ramp-up process and longer wavelengths of light can be applied at the end of the white light ramp-up process, or vice versa. The white light ramp-up process of step 302 is optional and can be omitted in some examples.

In some examples, the method 300 can be performed on a user while the user is asleep. As such, the white light ramp-up process at step 302 can be applied without requiring an additional time commitment from the user. This improves user-compliance. The white light ramp-up process of step 302 allows for the intensity of light applied to the user's eyelids to be increased over time, rather than increased immediately, which prevents the user from experiencing light exposure shock. Moreover, this may help avoid a user from waking up when receiving a dose of white light at step 304.

In step 304, a dose of white light is applied to the user's eyelids. As the dose of white light is applied to the user's eyelids, some of the white light is absorbed by the user's eyelid, and some of the white light is transmitted through the user's eyelids to the user's eyes. Light having red wavelengths (e.g., light with wavelengths in a range from about 600 nm to about 650 nm or in a range from about 600 nm to about 680 nm) has the highest transmittance through human eyelids, with a transmittance of about 5%. Thus, a dose of red light is applied to the user's eyes as the dose of white light is applied to the user's eyelids.

The dose of white light applied to the user's eyelids can include particular ranges of light wavelengths, which can be modifiable. As an example, different ranges of wavelengths of light can be applied to the user's eyelids based on a detected impact of the light on the user's eyes. The white light can be generated by a white LED or other light source, or a combination of colored light sources.

In some examples, the white light can be applied to the user's eyelids at a luminance of about 1,000 lux, about 10,000 lux, about 32,000 lux, in a range from about 1,000 lux to about 50,000 lux, in a range from about 1,000 lux to about 5,000 lux, in a range from about 5,000 lux to about 15,000 lux, in a range from about 25,000 lux to about 35,000 lux, or the like. The luminance of red light that passes through the user's eyelids to the user's eyes can be about 5% of the luminance of the white light that is applied to the user's eyelids. In some examples, the red light applied to the user's eyes can be applied at a luminance of about 50 lux, about 500 lux, about 1,600 lux, in a range from about 50 lux to about 2,500 lux, in a range from about 50 lux to about 250 lux, in a range from about 250 lux to about 750 lux, in a range from about 1,250 lux to about 1,750 lux, or the like.

The white light application of step 304 can have a duration in a range from about 2 minutes to about 3 minutes, about 3 minutes, or the like. The white light application of step 304 can have a duration in a range from about 2 minutes to about 4 minutes, from about 150 seconds to about 210 seconds, from about 1 minute to about 10 minutes, from about 1 minute to about 3 minutes, from about 2 minutes to about 5 minutes, or about 3 minutes.

Direct exposure of the eyes to red light can be associated with several potential health concerns. For example, direct exposure to red light can cause retinal burn. White light is cooler than red light, and thus application of white light rather than red light can avoid retinal burn and the like. Moreover, the user's eyelids help to diffuse incident light, which also prevents retinal burn and associated health concerns.

The method 300 includes performing a white light ramp-down process at step 306. The intensity of white light applied to the user's eyelids during the white light ramp-down process can be varied (e.g., decreased) continuously, or in a stepped manner. During the white light ramp-down process, the intensity of white light applied to a user's eyelids is decreased over a period of time. The white light ramp-down process can have a duration in a range from about 5 minutes to about 20 minutes, from about 8 minutes to about 12 minutes, from about 1 minute to about 30 minutes, about 5 minutes, about 10 minutes, about 15 minutes, or the like. In some examples, wavelengths of the white light applied to the user's eyelids can be varied during the white light ramp-down process of step 306. For example, shorter wavelengths of light can be applied at the beginning of the white light ramp-down process and longer wavelengths of light can be applied at the end of the white light ramp-down process, or vice versa. The white light ramp-down process of step 306 is optional and can be omitted in some examples.

In some examples, the method 300 can be performed on a user while the user is asleep. As such, the white light ramp-down process at step 306 can be applied without requiring an additional time commitment from the user. This improves user-compliance. The white light ramp-down process of step 306 allows for the intensity of light applied to the user's eyelids to be decreased over time, rather than decreased immediately, which prevents the user from experiencing light exposure shock. Moreover, this may encourage a user to continue sleeping after receiving the dose of white light at step 304.

In step 308, features of a user's eye are detected by a sensor. The sensor can be the same as or similar to the sensors 208, discussed above with respect to FIG. 2. Features of the user's eye can include the pupil diameter, choroidal thickness, axial length, scleral length, pressure applied from the white light therapy apparatus to the eye, choroidal and retinal vascular characteristics, and the like.

Although step 308 is illustrated as being performed after steps 302-306, step 308 can be formed at any appropriate times throughout the administration of white light therapy to a user. For example, measurements of step 308 can be taken before the ramp-up process of step 302, during the ramp-up process of step 302, after the ramp-up process of step 302 and before the white light exposure of step 304, during the white light exposure of step 304, after the white light exposure of step 304 and before the ramp-down process of step 306, during the ramp-down process of step 306, after the ramp-down process of step 306, or any combinations thereof. Any of the ramp-up process of step 302, the white light exposure of step 304, the ramp-down process of step 306, the number of white light doses administered per day or per week, and the like can be altered based on the measurements determined in step 308. In a specific example, step 308 is used to measure axial length of a user's eyes after the ramp-up process of step 302 (referred to as a baseline axial length), prior to the white light exposure of step 304; at the end of the white light exposure of step 304; and at intervals during the ramp-down process of step 306 (e.g., at 3, 6, and 9 minutes into the ramp-down process of step 306).

In some examples, step 308 can be used to measure a user's pupil size. It has been found that both of a user's eyes react synchronously to light, with both constricting and dilating by approximately the same amount when exposed to light. As such, one of a user's eyelids can be exposed to white light, while their other eye is open and not exposed to the white light. The diameter of the pupil of the user's open eye can be detected and measured to closely approximate the size of the pupil of the user's eye that is being exposed to white light. As such, the user's pupil size can be tracked as their eye is exposed to white light.

In some examples, step 308 can be used to measure various eye features as steps of the method 300 are carried out. In examples in which step 308 is used to measure pressure applied from a white light therapy apparatus to a user's eye, a pressure sensor can be used to measure pressure applied by the white light therapy apparatus. Tightness of a strap of the white light therapy apparatus can be adjusted based on the measured pressure applied to the user's eye. Optical coherence tomography (OCT), optical low coherence reflectometry (OLCR), or the like can be used to measure features of the user's eyes during the method 300, and specifically as steps of the method 300 are performed on the user. For example, OCT or OLCR can be used to measure the axial length of the user's eye during the method 300. In some examples, infrared light can be applied during the detection of step 308 in order to aid detecting features of the user's eye.

In step 310, the applied dose of white light is adjusted in response to the eye features detected in step 308. This can include altering the duration of steps 302-306, altering the wavelength of white light administered during steps 302-306, altering the intensity of white light administered during steps 302-306, altering the number of times the method 300 is administered per day or per week, ceasing administration of the method 300, extending the administration of the method 300 (e.g., for a longer period than initially proposed), or the like. Step 310 can be used to alter the administration of a current round of the method 300, or to alter the administration of subsequent rounds of the method 300.

The method 300 can be repeated a set number of times per day, a set number of times per week, and the like. For example, the method 300 can be repeated twice daily, five times weekly, for a period of at least a month. However, the method 300 can be repeated more or less times daily, weekly, and for a period sufficient to correct a user's eye conditions (e.g., a myopic condition). In some examples, all of the doses of white light administered through the method 300 can be administered while a user is sleeping. For example, two doses of white light can be administered to a user while the user is sleeping, with a period of at least about 4 hours in between doses. This allows for multiple doses to be administered while only requiring a single donning event by the user and increases user compliance relative to methods that require the user performing multiple actions. Moreover, the method 300 can be administered while the user is asleep, without requiring a time commitment from the user, further improving user compliance.

### EXAMPLES

### Example 1

The transmittance of light through the eyelid at various wavelengths can be estimated by trial. In a study, performed using 27 subjects, an equation is developed to estimate the log transmittance through the eyelid:

$$log ( {T}_{λ} ) = 0.7 \; 39 \times log ( {A}_{λ} ) + 1.3 \; 68 \times log ( {B}_{λ} ) + 2.6 \; 43 \times log ( {C}_{λ} ) + d log ( {D}_{λ} ) - log ( 0.828 ) \qquad Equation 1$$

where:
T_{λ} is the transmittance value at wavelength λ
A_{λ} is the transmittance value for deoxy-haemoglobin at wavelength λ
B_{λ} is the transmittance value for oxy-haemoglobin at wavelength λ
C_{λ} is the transmittance value for melanin at wavelength λ

Also, although melanin absorbance is primarily responsible for differences in external skin reflectance, the modeled values for its impact on eyelid transmittance does not vary much among subjects, hence skin pigmentation does not have a significant effect on the eyelid spectral transmittance. The final term of the equation, −log (0.828), represents a scattering constant for non-spectrally absorbing macromolecules.

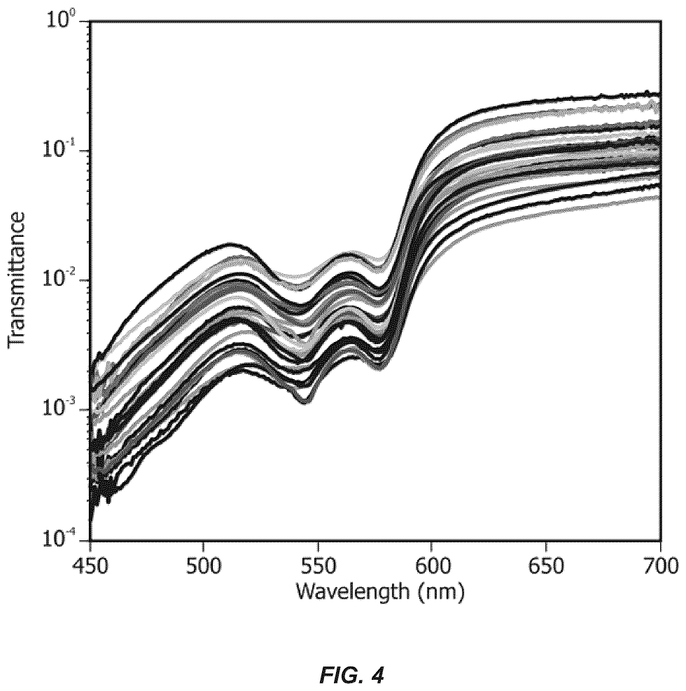

This relationship provides a calculated mean estimate of transmittance through the eyelid of 9% at 630 nm compared to 9% and 8.5% transmission, respectively, at 630 nm, as shown in FIG. 4. In this study, it is assumed that the mean transmittance levels through the eyelid for the 600-650 nm range was about 5%.

### Example 1: Illumination of Open and Closed Eyes

For the purposes of this study, a clinical slit lamp was used to provide illumination. As the slip lamp utilises a halogen lamp to provide illumination, to provide for a steady light flux over time, high power settings were used and allowed to equilibrate, and then a combination of neutral density filters were used to achieve the target illumination flux.

To ensure the maximum area of eyelid light exposure, participants were instructed to gently hold their left upper eyelid shut with the fingers adjacent to the lash base. The investigator then confirmed the light source wasn't being blocked by the participants fingers with the retina filled with the brightest light possible. The white light intensity was estimated to be 5% of the light intensity between 600 nm to 650 nm impinging onto the closed eyelid.

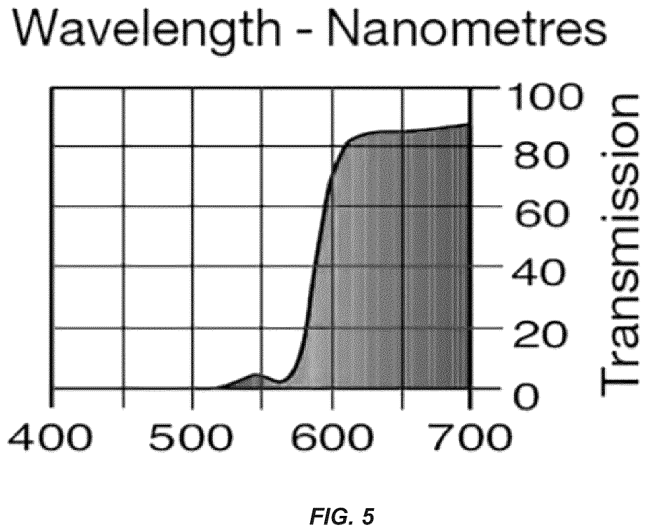

The red-light open eye conditions were created using an additional LEE 781 ‘Terry Red’ filter to the neutral density filters that removed nearly all of the short wavelength spectrum and had a relatively sharp cut-off near 600 nm, as shown in FIG. 5.

TABLE 1

| | | | Light intensity |
|---|---|---|---|
| | | Light | at the corneal |
| Condition | Light | Intensity (Lux) | plane (Lux) |
| 1 | Red (open eye) | Low - 50 | Low - 50 |
| 2 | | Mid - 560 | Mid - 560 |
| 3 | | High - 1600 (¹) | High - 1600 |
| 4 | White (closed eye) | Low - 1100 | Low - 55 |
| 5 | | Mid - 10,000 | Mid - 500 |
| 6 | | High - 32,000 | High - 1600 |

¹ Note 1: a red-light intensity of 1600 Lux is equivalent to that provided by a commercially available red-light therapy system prior art system.

### Study Subjects and Test Protocols:

The following studies were performed using six test subjects, 4 of whom were previously used in a separate red-light study. The participants had a mean age of 33±3 years with mean spherical equivalent refraction (SER) measurements of:
3 emmetropes with a mean SER of 0.16±0.38 D (range −0.25 to 0.50 D)
3 myopes with a mean SER of −2.04±1.09 D (range −1.12 to −3.25 D)

To match the commercially available RLRL therapy devices, the participant's left eye was exposed to either the red light (open eye) or white light (closed eye through the lids) test field for 3 minutes. The right eye was then occluded for both Lenstar and OCT measurements. Visits included exposure to two different light conditions. To minimise the potential cross over effects, a 30-minute break was provided between the last ocular measurements with the first light condition and the baseline measurement of the second light condition.

Axial length (AxL) and then choroidal thickness (ChT) of the left eye were measured at baseline and then immediately and 10 minutes after 3 minutes of exposure to full field light. Axial length was determined using a Lenstar system, which can determine various measurements of the ocular system using optical low coherence reflectometry (OLCR) for measurement of the axial length parameters of the eye. Choroidal thickness measurements were determined by measurement of the anatomical features of the anterior of the eye using Optical Coherence Tomography (OTC). The baseline measurements were collected after a 10-minute washout period of watching a grayscale movie at 4 m distance. The right eye was occluded during ocular measurements but remained opened for pupil recording during the left eye exposure to light.

The light conditions were tested in a randomised order on separate days and included:
White light through the lid at low, mid, and high intensities
Broadband red light through the open eye at low, mid, and high intensities

### Example 2

### Pupillary Contraction:

It is problematic to directly measure the pupil size of the exposed eye behind a closed eyelid. Back illumination with infrared through the skull has been effectively used on animals to measure pupil size through the eyelids, however there are no reports of this method being successfully tested in humans.

One way of gaining an accurate estimate of the direct pupil size behind a closed eyelid is to measure the pupil size of the fellow eye as the pupils of both eyes react synchronously to light (i.e., both constrict and dilate by approximately the same amount). As soon as light was incident on the closed eye, the pupil size of the fellow eye was measured using a handheld pupilometer. As part of an initial red-light study, it was determined that for each millimetre decrease in pupil size of the exposed eye there was a 0.93-millimetre decrease in the fellow eye.

Baseline pupil size of the fellow eye was measured in the experimental setup prior to light stimulation. As soon as light was incident on the eye, the pupil size of the fellow eye was measured again using a handheld pupilometer. As expected, there was a significant main effect of light intensity (p<0.001) upon percentage changes in pupil size from baseline.

The amount of pupil constriction was similar in the white light (through the lid) and red-light open conditions, however there was a significant main effect of the type of light (p<0.001) on the pupil constriction. This occurred because the overall constriction was greater with the open eye condition than the white light through the lid condition. This suggests that either the original estimate of about 5% transmission through the lids was too high (less light transmitting through the lids) or that the slight differences in wavelength reaching the retina (through the lids versus the broadband red filter in open eye) had an impact on the pupil constriction. Either or both factors may have contributed to the difference in pupil constriction (5-10%).

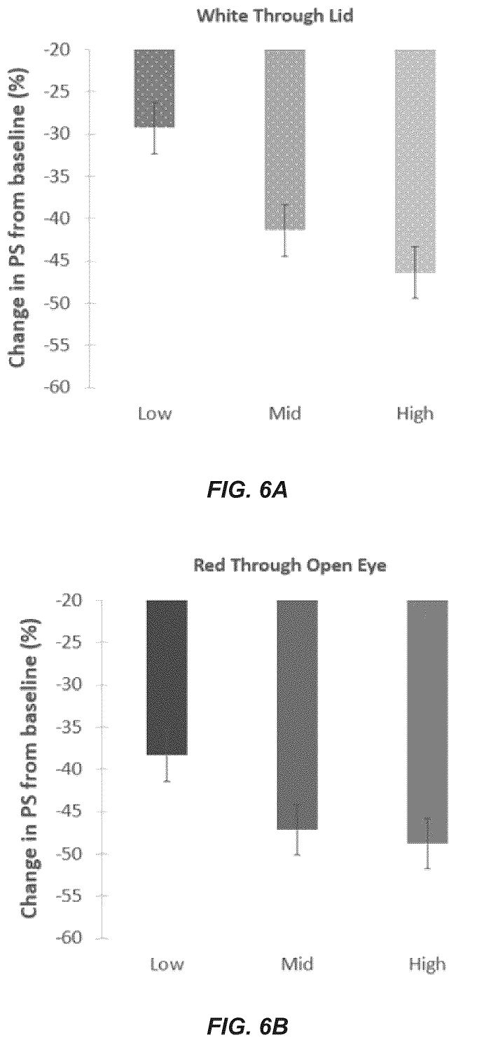

As shown in FIGS. 6A and 6B, pupil contractions for white light transmitted through the eyelid (low, mid and high white light intensities) were observed to be 9.2%, 20.8% and 25.4% respectively from their baseline values, and for red light illuminating open eyes (low, mid and high red light intensities), pupil contractions of 17.7%, 26.32% and 28.5% were observed.

### Example 3

### Axial Length:

The mean value for baseline Axial Length (AxL) was 24.00±0.58 mm. Baseline AxL did not vary significantly with light conditions (p=0.399) or light intensity (p=0.942). Also, there was no significant light by intensity interaction on measures of baseline AxL (p=0.445), suggesting that:
AxL measurements were not confounded by responses to the preceding light condition
Potential light driven changes in AxL were decayed within 40 minutes after the 3-minute light exposure
Light condition had a significant impact upon AxL changes (p=0.016), while no significant effects were found for light intensity, time, or their interactions (all p>0.05)

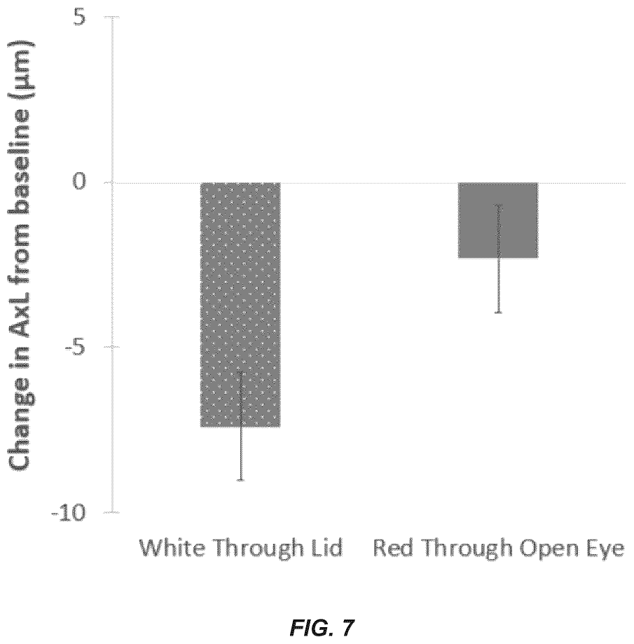

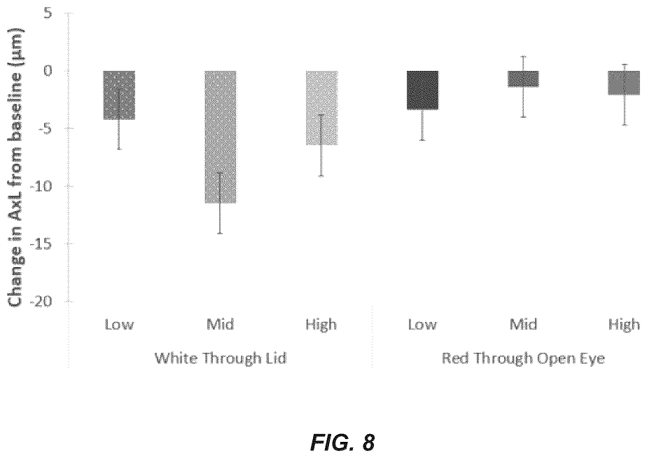

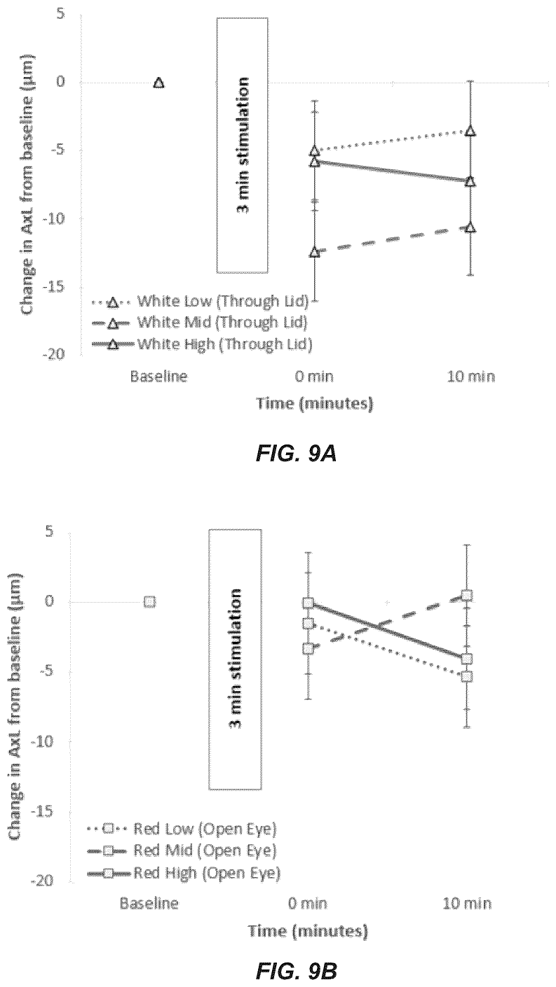

Main effect of light (p=0.016), changes averaged across three levels of light intensity and across 0- and 10-minutes post exposure times are shown in FIG. 7. Light by intensity interaction was p=0.186 for axial length changes averaged across 0-minute and 10-minutes post exposure times (see FIG. 8) with the greatest effect being seen for white light illumination with a medium intensity. Light by intensity by time interaction was not significant for 0 and 10 minutes post-exposure p=0.764 (FIGS. 9A and 9B) respectively.

### Example 4

### Subfoveal Choroidal Thickness:

The mean (±SEM) value for baseline subfovial choroidal thickness (SFCT) was 287±26 μm. Whilst baseline SFCT did not vary significantly with light conditions (p=0.369), there was a significant main effect of intensity (p=0.013) and a significant intensity by light interaction (p=0.04). Post hoc tests revealed that the main reason underlying these significant effects was a significantly thinner baseline SFCT in the White Mid condition compared to White High condition.

Given that the order of testing conditions was randomised, and the White Mid condition was never preceded by the White High condition in a single visit for any participant, it is unlikely that the significant light by intensity interaction could be attributed to any carryover effects. However, the statistical analysis incorporated both absolute changes in SFCT and percentage changes in SFCT from baseline to mitigate the potential impact of baseline SFCT variability on the observed light-induced changes.

To assess these, a linear mixed-model (LMM) statistical analysis was applied. Linear mixed models are an extension of simple linear models to allow both fixed and random effects and are particularly used when there is non independence in the data, such as arises from a hierarchical structure.

A linear mixed model (LMM) analysis of absolute changes in SFCT from baseline revealed a significant light by intensity interaction (p=0.005). LMM analysis on percentage changes in SFCT also revealed a significant main effect of intensity (p=0.033) and a significant light by intensity interaction (p=0.004). Given the similar findings with both statistical approaches, only the outcomes from the absolute changes in SFCT are presented.

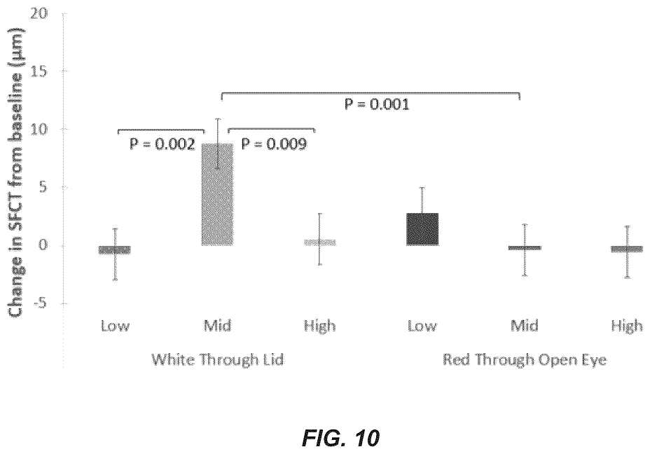

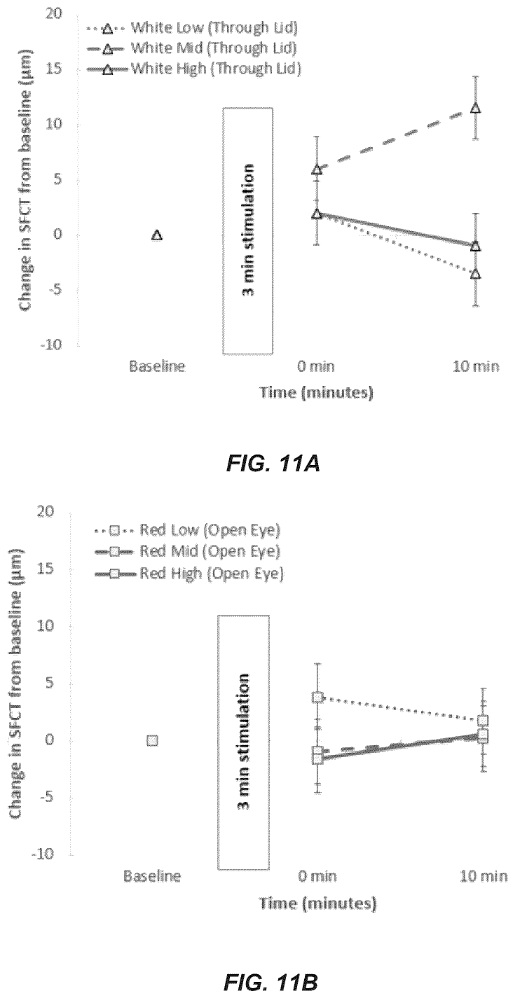

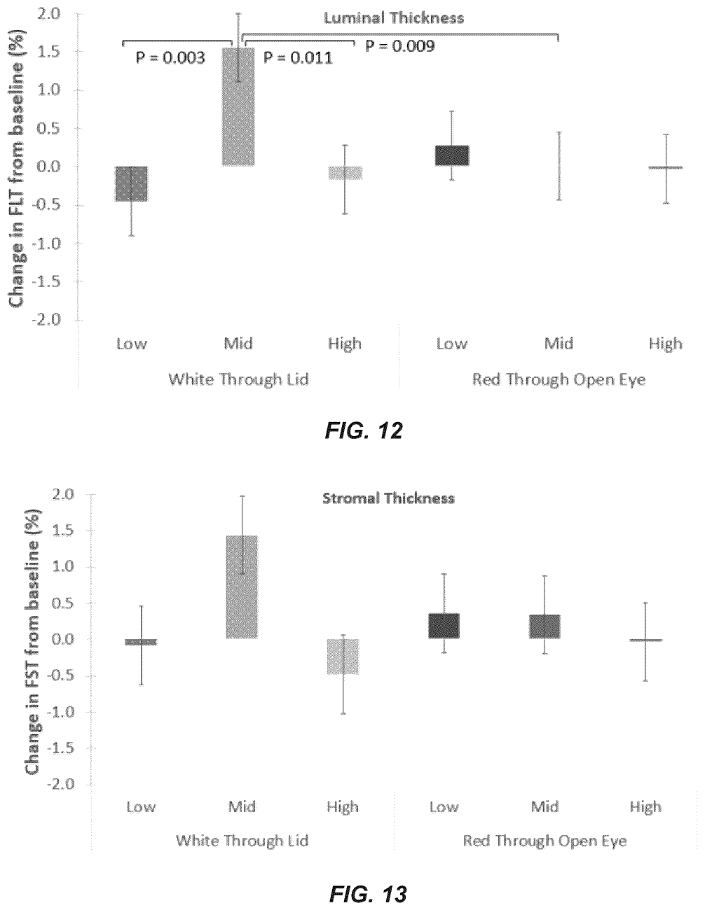

A significant light by intensity interaction (p=0.005) was found for SFCT changes averaged across 0- and 10-minutes post exposure times (see FIG. 10). Light by intensity by time interaction was not significant (p=0.764) (see FIGS. 11A and 11B). The LMM analysis on percentage changes in foveal luminal thickness (FLT) revealed a significant light by intensity interaction (p=0.019) (see FIG. 12).

The LMM analysis on percentage changes in foveal stromal thickness (FST) also revealed a light by intensity interaction (p=0.139) that approached significance (see FIG. 12).

### Example Conclusions

The aim of these studies was to investigate the short-term effects of white light exposure through the eyelids in comparison to direct broadband red light exposure on the axial length of the eye as well as vascular and thickness changes in the choroid.

The light levels used to deliver the white light through the lids were chosen to produce an estimated light level at the corneal plane of 1600 lux (equivalent to Eyerising), 500 lux and 55 lux. A broadband red light was used as a control condition with an open eye with similar light intensity levels to the white light through the lids conditions (1600 lux, 500 lux and 55 lux).

The changes in pupil size with the white light through the lids (measured from the fellow eyes) compared with the broadband red light in the open eye were slightly greater in the open eye condition. This suggests that the retinal illuminance levels were slightly higher in the open eye red filter condition.

The baseline axial length and choroidal thickness measured before each condition were similar and not significantly different across time. This indicates that the 40 minutes delay used in the protocol between the testing of the six different light conditions was sufficient for the effects to decay from the last tested light condition.

The changes in axial length and subfoveal choroidal thickness after exposure to the six light conditions followed a similar pattern and were statistically correlated.

The high and low intensity white light through the lid and all the broadband red (low, mid and high) intensities produced minimal changes in axial length and choroidal thickness. However, the white light through the lid mid-intensity (10 000 lux at the eyelids and ˜500 lux through the lids at the cornea) was consistently the most effective at reducing axial length and thickening the choroid, producing greater effects than seen with open-eye red light illumination, such as used in prior art devices.

While the examples above have been described with reference to specific types of ocular lenses, feature shapes, feature materials, layers, and other parameters, any appropriate type of parameter may be incorporated into the ocular lenses in accordance with the principles of the present disclosure. Thus, any number of features, shapes, or layers may be used in accordance with the principles described herein. Further, multiple types of materials with differing optical refractive characteristics may be used to make the ocular lenses. Further, the ocular lenses may be made with different material to achieve optimal bonding, spacing, adhesion, optics, or other types of characteristics.

The terms recited in the claims should be given their ordinary and customary meaning as determined by reference to relevant entries in widely used general dictionaries and/or relevant technical dictionaries, commonly understood meanings by those in the art, and the like, with the understanding that the broadest meaning imparted by any one or combination of these sources should be given to the claim terms (e.g., two or more relevant dictionary entries should be combined to provide the broadest meaning of the combination of entries) subject only to the following exceptions: (a) if a term is used in a manner that is more expansive than its ordinary and customary meaning, the term should be given its ordinary and customary meaning plus the additional expansive meaning, or (b) if a term has been explicitly defined to have a different meaning by reciting the term followed by the phrase “as used herein shall mean” or similar language (e.g., “herein this term means,” “as defined herein,” “for the purposes of this disclosure the term shall mean,” or the like).

References to specific examples, use of “i.e.,” use of the word “invention,” and the like, are not meant to invoke exception (b) or otherwise restrict the scope of the recited claim terms. Other than situations where exception (b) applies, nothing contained herein should be considered a disclaimer or disavowal of claim scope.

The subject matter recited in the claims is not coextensive with and should not be interpreted to be coextensive with any particular embodiment, feature, or combination of features shown herein. This is true even if only a single embodiment of the particular feature or combination of features is illustrated and described herein. Thus, the appended claims should be given their broadest interpretation in view of the prior art and the meaning of the claim terms.

As used herein, spatial or directional terms, such as “left,” “right,” “front,” “back,” and the like, relate to the subject matter as it is shown in the drawings. However, it is to be understood that the described subject matter may assume various alternative orientations and, accordingly, such terms are not to be considered as limiting.

Articles such as “the,” “a,” and “an” can connote the singular or plural. Also, the word “or” when used without a preceding “either” (or other similar language indicating that “or” is unequivocally meant to be exclusive—e.g., only one of x or y, or the like) shall be interpreted to be inclusive (e.g., “x or y” means one or both x or y).

The term “and/or” shall also be interpreted to be inclusive (e.g., “x and/or y” means one or both x or y). In situations where “and/or” or “or” are used as a conjunction for a group of three or more items, the group should be interpreted to include one item alone, all of the items together, or any combination or number of the items. Moreover, terms used in the specification and claims such as have, having, include, and including should be construed to be synonymous with the terms comprise and comprising.

Unless otherwise indicated, all numbers or expressions, such as those expressing dimensions, physical characteristics, and the like used in the specification (other than the claims) are understood as modified in all instances by the term “approximately.” At the very least, and not as an attempt to limit the application of the doctrine of equivalents to the claims, each numerical parameter recited in the specification or claims which is modified by the term “approximately” should at least be construed in light of the number of recited significant digits and by applying ordinary rounding techniques.

All ranges disclosed herein are to be understood to encompass and provide support for claims that recite any and all subranges or any and all individual values subsumed therein. For example, a stated range of 1 to 10 should be considered to include and provide support for claims that recite any and all subranges or individual values that are between and/or inclusive of the minimum value of 1 and the maximum value of 10; that is, all subranges beginning with a minimum value of 1 or more and ending with a maximum value of 10 or less (e.g., 5.5 to 10, 2.34 to 3.56, and so forth) or any values from 1 to 10 (e.g., 3. 5.8. 9.9994, and so forth).

What is claimed is:

1. A method for controlling axial length of a user's eye, the method comprising:
applying a dose of white light to the user's eyelid;
detecting an effect of the applied white light on the user's eye; and
adjusting the applied white light based on the detected effect.

2. The method of claim 1, wherein applying the dose of white light to the user's eyelid applies a dose red light to the user's eye having a wavelength in a range from 600 nm to 650 nm.

3. The method of claim 1, wherein the detected effect of the applied white light is the axial length of the user's eye.

4. The method of claim 1, further comprising applying a white light with a ramped-up intensity prior to applying the dose of white light.

5. The method of claim 1, further comprising applying a white light with a ramped-down intensity subsequent to applying the dose of white light.

6. The method of claim 1, further comprising detecting a diameter of a pupil of the user's other eye as the white light is applied to the user's eye.

7. The method of claim 6, further comprising adjusting the applied white light based on the detected pupil diameter of the user's eye.

8. The method of claim 1, further comprising detecting pressure applied to the user's eye from a white light source during the application of the white light.

9. The method of claim 8, further comprising adjusting a tightness of a strap configured to retain the white light source on the user's head in response to the detected pressure.

10. The method of claim 1, wherein the white light is applied to the user while the user is in a sleeping state.

11. The method of claim 1, wherein the applied dose of white light has an illuminance in a range from 30,000 lux to 35,000 lux.

12. The method of claim 1, wherein the applied dose of white light is applied for a duration in a range from 2 minutes to 3 minutes.

13. A system configured to apply red light to a user's eye through the user's eyelid, the system comprising:
a white light source;
a sensor configured to detect an axial length of a user's eye; and
a controller configured to vary the white light source based on the detected axial length of the user's eye.

14. The system of claim 13, wherein the sensor uses optical low coherence reflectometry to detect the axial length of the user's eye.

15. The system of claim 13, wherein the sensor uses optical coherence tomography (OCT) to detect the axial length of the user's eye.

16. The system of claim 13, wherein the sensor is configured to direct infrared light towards the user's eye.

17. The system of claim 13, further comprising:
a device housing that houses the white light source, the sensor, and the controller; and
a strap configured to retain the system on the user's head.

18. The system of claim 13, further comprising a pressure sensor configured to detect pressure applied by the device housing to the user's eyes, wherein the strap is adjustable based on the pressure detected by the pressure sensor.

19. An apparatus comprising:
a device housing, the device housing comprising:
a white light source configured to apply a white light to a user's eyelid; and
a sensor configured to detect a characteristic of an eye; and
a strap coupled to the device housing and configured to retain the device housing on a user's head.

20. The apparatus of claim 19, wherein:
the sensor is a pressure sensor;
the sensor is configured to detect pressure applied to an eye by the apparatus; and
the strap is configured to be adjusted based on the detected pressure.
